(19) United States
(12) Patent Application Publication
(10) Pub. No.: US 2017/0106172 A1
(43) Pub. Date: Apr. 20, 2017

(54) GUIDE WIRE

(71) Applicant: TERUMO KABUSHIKI KAISHA, Tokyo (JP)

(72) Inventors: Yasunao OOTANI, Ito-shi (JP); Yutaka TANO, Fujinomiya-shi (JP); Shuuhei INOUE, Fujinomiya-shi (JP); Jo FUJIKI, Mishima-shi (JP); Kayo KAMBARA, Sunto-gun (JP)

(73) Assignee: TERUMO KABUSHIKI KAISHA, Shibuya-ku, Tokyo (JP)

(21) Appl. No.: 15/395,447

(22) Filed: Dec. 30, 2016

Related U.S. Application Data
(63) Continuation of application No. PCT/JP2015/074302, filed on Aug. 27, 2015.

(30) Foreign Application Priority Data
Sep. 26, 2014 (JP) 2014-196648

(51) Int. Cl. A61M 25/09

(52) U.S. Cl. CPC A61M 25/09; A61M 2025/09083; A61M 2025/09133

(57) ABSTRACT

A guide wire is disclosed, which includes a core portion formed of an elongated object having flexibility, in which the core portion includes a main body portion formed on a proximal side, a flat plate portion formed on a distal side, and a transition portion which connects the main body portion and the flat plate portion, and in which at least one groove portion extending in a direction different from a length direction is formed on a slope of the transition portion in the length direction. In addition, the guide wire includes a coil portion disposed so as to cover the distal side of the core portion.

### CROSS-REFERENCES TO RELATED APPLICATIONS

This application is a continuation of International Application No. PCT/JP2015/074302 filed on Aug. 27, 2015, and which claims priority to Japanese Patent Application No. 2014-196648 filed on Sep. 26, 2014, the entire contents of both, which are incorporated herein by reference.

### TECHNICAL FIELD

The present disclosure relates to a guide wire used when guiding a catheter into a lumen in a living body, in particular, a blood vessel.

### BACKGROUND DISCUSSION

The guidewire is used when guiding a catheter which is used for treatment of a site in which it is difficult to perform a surgical operation, for example, percutaneous transluminal coronary angioplasty (PTCA) or treatment which is aimed to be less invasive to the human body, or used in tests such as cardioangiography, into a blood vessel. PTCA is a treatment method for dilating a stenosed site of a coronary artery with a balloon to secure a blood flow path.

In PTCA, a balloon catheter is guided to a stenosed site by inserting a guide wire into the vicinity of the stenosed site of a blood vessel in a state in which a distal portion of the guide wire protrudes from a distal portion of the balloon catheter. At that time, it is necessary for the guide wire to select and pass through a meandering or bifurcated blood vessel, or a stenosed blood vessel. In addition, it is necessary to widen or penetrate deposits such as cholesterol constituting the stenosed site using a pushing force of the guide wire in the stenosed site. Accordingly, flexibility (blood vessel followability) for following the shape of a blood vessel and for preventing damage to a blood vessel wall, and excellent pushing performance (pushability) which can help ensure effective transmission of a pushing force from the operator's hand side (proximal portion) to a distal portion are required for the guide wire used for PTCA.

In addition, in PTCA, in some cases, reshaping at a distal end is performed before inserting the guide wire into the blood vessel in order to make the guide wire follow the bent and bifurcated blood vessel. Specifically, for example, a surgeon can perform the reshaping by bending the distal portion of the guide wire into a predetermined shape (for example, J shape) using fingers in accordance with the shape of the bifurcated blood vessel or the like. Accordingly, it can be necessary for the guide wire to easily perform such reshaping at a distal end.

In the related art, a guide wire including the following configuration has been proposed in International Publication No. WO/2009/126656 for PTCA. The guide wire in International Publication No. WO/2009/126656 includes a core portion formed of an elongated object; and a coil, which is provided so as to cover a distal side of the core portion. The core portion has a flat plate portion, which is formed to have a plate width equal to or more than twice the plate height (plate thickness), on the distal side.

In the guide wire of International Publication No. WO/2009/126656, the distal portion of the guide wire becomes flexible because a flat plate portion with a thin plate thickness is provided on the distal side of the core portion, which is expected to improve safety and blood vessel followability to some degree. However, the guide wire in International Publication No. WO/2009/126656 includes a round rod-like main body portion which is formed on a proximal side and has a circular shape in transverse cross section; a flat plate portion which is formed on a distal side and has a rectangular shape in transverse cross section; and a transition portion which connects the main body portion and the flat plate portion. Accordingly, in the guide wire of International Publication No. WO/2009/126656, the transverse cross sectional shape greatly changes from the transition portion over the flat plate portion. Therefore, the physical properties (in particular, for example rigidity) also greatly change from the transition portion over the flat plate portion.

In such a guide wire, when the proximal portion of the guide wire is rotated in order to make the guide wire pass a meandering or bifurcated blood vessel, the flat plate portion is twisted or is buckled in the vicinity of a boundary between the transition portion and the flat plate portion. As a result, rotary torque at the proximal portion of the guide wire is not effectively transmitted from the proximal portion to the distal portion. Therefore, the distal portion of the guide wire does not face an intended direction and blood vessel followability of the guide wire is decreased. In addition, torquability is decreased due to twisting of the flat plate portion when the proximal portion of the guide wire is rotated in order to advance the guide wire in a stenosed site, or that pushability and trackability (properties of transmitting a rotational force, which is applied to the guide wire at the proximal portion, to the distal portion) of the guide wire without effective transmission of a pushing force of the proximal portion of the guide wire to the distal portion due to buckling of the flat plate portion in the vicinity of a boundary between the transition portion and the flat plate portion.

### SUMMARY

In accordance with an exemplary embodiment, a guide wire is disclosed having excellent blood vessel followability, pushability, and trackability.

A guide wire is disclosed, which includes a core portion formed of an elongated object having flexibility, in which the core portion includes a main body portion formed on a proximal side, a flat plate portion formed on a distal side, and a transition portion which connects the main body portion and the flat plate portion, and in which at least one groove portion extending in a direction different from a length direction is formed on a slope of the transition portion in the length direction.

In addition, it can be preferable that the guide wire according to the present disclosure includes a coil portion which is disposed so as to cover the distal side of the core portion and is obtained by forming strands in a spiral shape, and the core portion and the coil portion are fixed to each other on the distal side. In addition, it can be preferable that the guide wire according to the present disclosure includes a resin covering portion which is formed so as to cover the distal side (or portion) of the core portion and is made of a resin material.

According to the configuration, a portion with a thin plate thickness is disposed on the distal side of the guide wire because the flat plate portion is provided on the distal side of the core portion. Therefore, rigidity at a distal portion of the guide wire is decreased and flexibility of the guide wire at the distal portion is improved. In addition, since the rigidity at the distal portion of the guide wire is decreased, reshaping of the distal portion of the guide wire can be easily performed in accordance with the shape of, for example, a bifurcated blood vessel. In addition, significant change in the transverse cross sectional shape and significant change in the rigidity from the transition portion over the flat plate portion can be prevented because at least one groove portion is formed in the transition portion, which connects the main body portion and the flat plate portion of the core portion. Accordingly, twisting of the flat plate portion or buckling in the vicinity of a boundary between the transition portion and the flat plate portion can be suppressed when the guide wire passes a meandering or bifurcated blood vessel or when the guide wire advances in a stenosed site. As a result, rotary torque of a proximal portion of the guide wire can be effectively transmitted to the distal portion, and therefore, the distal portion of the guide wire can face an intended direction. In addition, pushing force of the proximal portion of the guide wire can be effectively transmitted to the distal portion of the guide wire.

In addition, in the guide wire according to the present disclosure, it can be preferable that at least one groove portion extending in the direction different from the length direction is formed on at least an upper surface or a lower surface of the flat plate portion in the length direction.

According to the configuration, the rigidity of the flat plate portion is further decreased because the groove portion is formed in the flat plate portion. Therefore, the rigidity at the distal portion of the guide wire at which the flat plate portion is disposed is decreased and the flexibility of the guide wire at the distal portion can be further improved.

According to the guide wire of the present disclosure, the flexibility of the guide wire on the distal side is improved and the change in the rigidity of a distal flexible portion is decreased. Therefore, blood vessel followability of the guide wire can be improved. In addition, since the buckling of the core portion can be suppressed, rotary torque or pushing force at the proximal portion of the guide wire can be effectively transmitted to the distal portion. Therefore, blood vessel followability, pushability, and trackability of the guide wire can be improved.

A guide wire is disclosed formed of an elongated object having flexibility, the guide wire comprising: a main body portion formed on a proximal side, the main body portion has a large-diameter portion having a constant outer diameter from a proximal side to a distal side, a first tapered portion of which an outer diameter is decreased toward the distal side, a middle-diameter portion having a constant outer diameter, a second tapered portion of which an outer diameter is decreased toward the distal side; and a small-diameter portion having a constant outer diameter; a flat plate portion formed on a distal side, the flat plate portion having at least one groove portion extending in a direction different from a length direction of the flat plate portion on at least an upper surface or a lower surface of the flat plate portion in the length direction of flat plate portion; and a transition portion which connects the main body portion and the flat plate portion, the transition portion having at least one groove portion extending in a direction different from a length direction of the transition portion on a slope of the transition portion in the length direction of the transition portion.

A guide wire is disclosed, the guide wire comprising: a core portion formed of an elongated object having flexibility, wherein the core portion includes a main body portion formed on a proximal side, a flat plate portion formed on a distal side, and a transition portion which connects the main body portion and the flat plate portion, and at least one groove portion extending orthogonal to a length direction of the transition portion on a slope of the transition portion.

### BRIEF DESCRIPTION OF DRAWINGS

FIG. 1 is a partial longitudinal sectional view showing a first exemplary embodiment of a guide wire of the present disclosure.

FIG. 2 is a side view of a core portion of the guide wire on a distal side shown in FIG. 1.

FIG. 3 is a plan view of the core portion of the guide wire on the distal side shown in FIG. 1.

FIG. 4 is an end surface view of the core portion taken along line IV-IV shown in FIG. 3.

FIG. 5 is an end surface view of the core portion taken along line V-V shown in FIG. 3.

FIG. 6 is a side view of a core portion on a distal side, which shows a second exemplary embodiment of a guide wire of the present disclosure.

FIG. 7 is a plan view of the core portion on the distal side shown in FIG. 6.

FIG. 8 is a partial longitudinal sectional view showing a third exemplary embodiment of a guide wire of the present disclosure on a distal side of the wire.

### DETAILED DESCRIPTION

A first exemplary embodiment of a guide wire according to the present disclosure will be described in detail while referring to drawings. Note that, in the present disclosure, a distal side refers to a side on which a guide wire is inserted into a blood vessel, and a proximal side refers to a side on which, for example, a surgeon operates the guide wire.

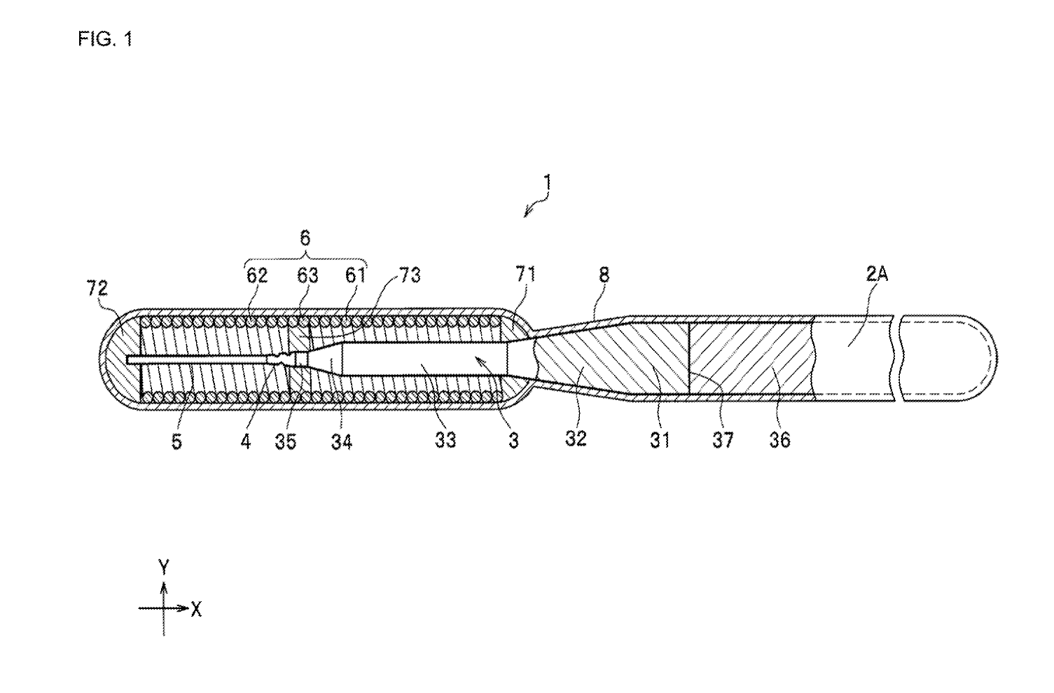

As shown in FIG. 1, a guide wire (hereinafter, referred to as a wire) 1 is an elongated object including a core portion 2A which can include a main body portion 3, a transition portion 4, and a flat plate portion 5. Groove portions 41 and 42 are formed in the transition portion 4. The total length of the wire 1 is not particularly limited, and is preferably, for example, 200 mm to 5000 mm. In addition, it can be preferable that the wire 1 includes a coil portion 6 disposed so as to cover a distal side (or portion) of the core portion 2A, and in which the core portion 2A and the coil portion 6 are fixed to each other on the distal side (or portion). As the fixing method, a fixation material (fixation portion) 72 such as solder (brazing material) or an adhesive material is preferably used for the fixing, and the fixation portion 72 may be formed through welding. Hereinafter, each configuration will be described.

As shown in FIGS. 1 to 3, the core portion 2A is formed of an elongated object having flexibility. The core portion 2A is preferably made of an elastic metal material such as Ni—Ti alloy or stainless steel in consideration of the flexibility and the strength of the wire 1. The core portion 2A sequentially includes the main body portion 3, the transition portion 4, and the flat plate portion 5 from a proximal side to the distal side, and at least one of the groove portion 41 or the groove portion 42 is formed in the transition portion 4. Note that the groove portion 42 may not be formed.

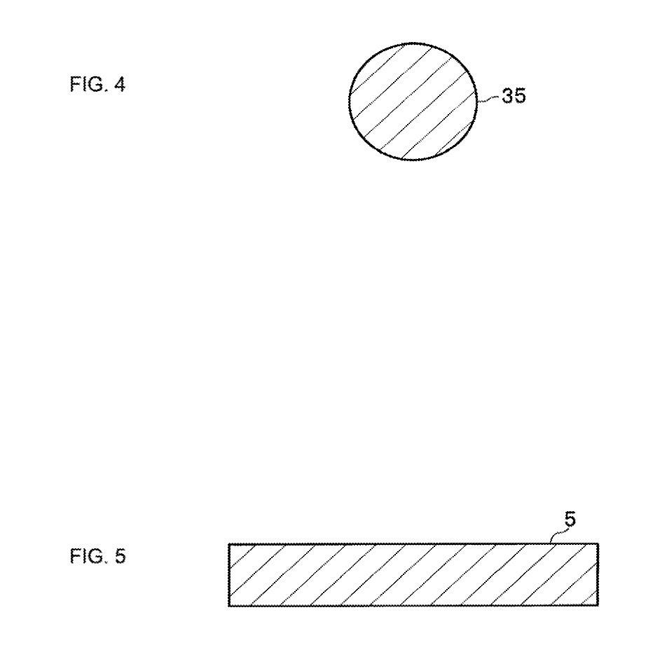

As shown in FIGS. 1 to 3, the main body portion 3 is formed of an elongated object with a bar shape (non-plate shape). In accordance with an exemplary embodiment, it can be preferable that the transverse cross sectional shape (which is a YZ-axis plane and a cross section perpendicular to a length direction) of the main body portion 3 is substantially a circular shape (refer to FIG. 4). In addition, it can be preferable that the main body portion 3 includes a large-diameter portion 31 having a constant outer diameter from the proximal side to the distal side; a first tapered portion 32 of which the outer diameter is decreased toward the distal side; a middle-diameter portion 33 having a constant outer diameter, a second tapered portion 34 of which the outer diameter is decreased toward the distal side; and a small-diameter portion 35 having a constant outer diameter.

Two tapered portions of the first tapered portion 32 and the second tapered portion 34 are described above as tapered portions formed between portions (between the large-diameter portion 31 and the middle-diameter portion 33, and between the middle-diameter portion 33 and the small-diameter portion 35) which have a constant diameter. However, the number of the tapered portions is not limited to two, and at least one tapered portion may be formed. In addition, a large-diameter portion 36 which has the same outer diameter as that of the large-diameter portion 31 and has a constituent material different from that of the large-diameter portion 31 may be joined to the large-diameter portion 31 in a joint portion (welded portion) 37. The joining method is not particularly limited, but examples thereof include butt resistance welding such as friction pressure welding, spot welding using a laser, or upset welding, and joining using a tubular joint member.

As shown in FIGS. 1 to 3, the flat plate portion 5 provides flexibility to the wire 1 (core portion 2A) and is formed of an elongated plate-shaped flat plate having a rectangular shape in transverse cross section (refer to FIG. 5) so as to facilitate reshaping of a distal portion of the wire at the distal end. The flat plate portion 5 preferably has, for example, a plate length of 1 mm to 30 mm, a plate width of 0.1 mm to 0.5 mm, and a plate width of 0.01 mm to 0.06 mm. In addition, the plate width of the flat plate portion 5 may be increased or decreased toward the distal side, and the plate thickness may also be increased or decreased toward the distal side.

In addition, the distal side of the flat plate portion 5 can be fixed to the coil portion 6 using the fixation material (fixation portion) 72. In addition, the flat plate portion 5 can be preferably produced together with the transition portion 4 to be described below by pressing the distal side of the bar-shaped main body portion 3, preferably the distal side of which the diameter is reduced, using, for example, a mold. Note that since the transverse cross sectional shape of the flat plate portion 5 can be produced through the pressing, both ends of the flat plate portion 5 can be slightly rounded and have an approximately rectangular shape in transverse cross section. However, the roundness of the both ends are omitted in FIG. 5 for the convenience of description.

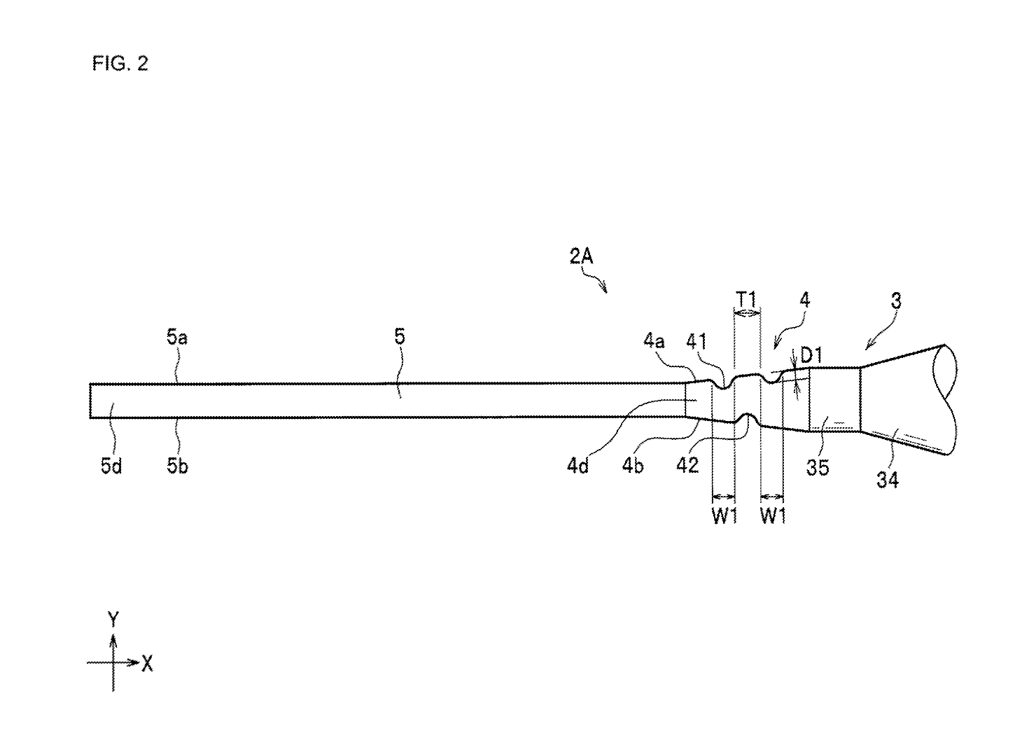

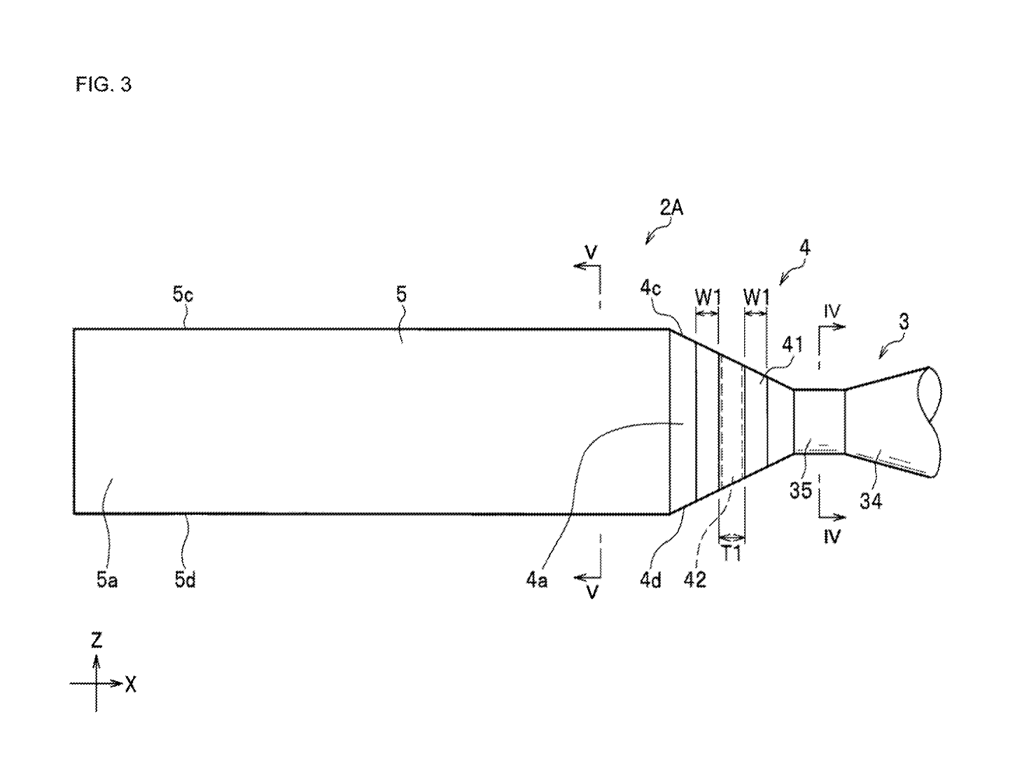

As shown in FIGS. 2 and 3, the transition portion 4 is a portion which connects the main body portion 3 and the flat plate portion 5 and which is gradually changed from a circular shape in transverse cross section (refer to FIG. 4) to the rectangular shape in transverse cross section (refer to FIG. 5) from the proximal side toward the distal side. The length of the transition portion 4, for example, is preferably 1 mm to 10 mm. In addition, the transition portion 4 has four slopes (edges) 4a, 4b, 4c, and 4d, which are connected to surfaces of the flat plate portion 5. At least one of the groove portion 41 or the groove portion 42 extending in a direction different from the length direction can be formed on at least one slope in the length direction. Note that the groove portion 42 may not be formed. The groove portions 41 and 42 are preferably produced by pressing a mold having a surface of a mold, in which convex portions with a shape similar to the groove portions 41 and 42 are formed, on the slope of the transition portion 4.

As shown in FIGS. 2 and 3, the groove portion 41 is formed on the slope 4a on the same surface side as an upper surface 5a of the flat plate portion 5. Note that, although not shown in the drawing, the groove portion 41 may be formed on the slope 4b or the slopes 4c and 4d on the same surface sides as a lower surface 5b or side surfaces 5c and 5d of the flat plate portion 5. Although not shown in the drawing, the groove portion 41 may be continuously formed on at least two slopes out of peripheral surfaces consisting of the slope 4a to the slope 4d.

The direction in which the groove portion 41 is formed is not particularly limited, but is preferably a direction orthogonal to the length direction as shown in FIG. 3. Note that, although not shown in the drawing, the groove portion 41 may be formed in a direction inclined to the direction orthogonal to the length direction at a predetermined angle. Furthermore, the planar shape of the groove portion 41 is preferably a linear shape as shown in FIG. 3, but may be a polygonal line shape or a curved shape.

In accordance with an exemplary embodiment, it is preferable that the transverse cross sectional shape of the groove portion 41 formed in the transition portion 4 is an approximately semi-circular shape. However, the transverse cross sectional shape of the groove portion 41 may be other shapes, for example, an approximately U-shape, an approximately V-shape, or an approximately rectangular shape. The number of groove portions 41 is preferably, for example, 1 to 100. The groove width W1 of the groove portion 41 is preferably, for example, 0.001 mm to 3 mm. The groove depth D1 of the groove portion 41 is preferably, for example, 0.005 mm to 0.05 mm. In addition, in a case of forming a plurality of groove portions 41, the groove width W1 is preferably formed to be constant, but may be formed so as to be increased or decreased toward the distal side. The groove depth D1 is also preferably formed to be constant, but may be formed so as to be increased or decreased toward the distal side.

An interval T1 of adjacent groove portions 41 is preferably, for example, 0.005 mm to 3 mm. The plurality of groove portions 41 are preferably formed at even (or equal) intervals, but may be formed so that the interval T1 increases or decreases toward the distal side. In addition, the plurality of groove portions 41 may have an interval T1 of, for example, 0 mm, that is, may be continuously formed. Furthermore, the groove portions 41 which have been continuously formed may be formed in a part or all of the slopes 4a, 4b, 4c, and 4d of the transition portion 4.

As shown in FIGS. 2 and 3, in the transition portion 4, it is preferable that the groove portion 41 is formed on the slope 4a and the groove portion 42 is formed on the slope 4b on the same surface side as the lower surface 5b of the flat plate portion 5. In addition, it is preferable that the groove portion 41 and the groove portion 42 are mutually alternately disposed in the length direction of the transition portion 4. However, the groove portion 41 and the groove portion 42 may be disposed at the same position as each other. In addition, although not shown in the drawing, in a case where the groove portion 41 is formed on the slope 4b, the groove portion 42 is formed on the slope 4a. In addition, although not shown in the drawing, in a case where the groove portion 41 is formed on one of the slopes 4c and 4d, the groove portion 42 is formed on one of the slopes 4d or 4c, which is on a side opposite to the slope on which the groove portion 41 is formed.

Although not shown in the drawing, in a case where the groove portions 41 are continuously formed on at least two slopes on the peripheral surface consisting of the slopes 4a to 4d, the groove portions 42 may be disposed and formed alternately with the groove portion 41 on slopes on which the groove portion 41 has not been formed.

The formation direction, the planar shape, the transverse cross-sectional shape, the number of grooves, the groove width, and the groove depth of the groove portion 42, and the interval between adjacent groove portions 42 are the same as those of the groove portion 41, and therefore, the description thereof will not be repeated. In addition, the formation direction of the groove portion 42 is preferably the same as that of the groove portion 41, but may be different from that of the groove portion 41.

Although not shown in the drawing, in a case where a plurality of groove portions 41 are formed, other groove portions which communicate with two or more arbitrary groove portions 41 in the length direction of the transition portion 4 may be formed. In addition, even in a case where a plurality of groove portions 42 are formed, other groove portions which communicate with two or more arbitrary groove portions 42 in the length direction of the transition portion 4 may also be formed.

In the wire 1 of the present disclosure, forming of the groove portions 41 and 42 suppresses great change in the transverse cross sectional shape from the transition portion 4 over the flat plate portion 5, and therefore, relatively large change in the rigidity can also be suppressed. As a result, there is no case where the flat plate portion 5 is twisted when using the wire 1 or is buckled in the vicinity of a boundary between the transition portion 4 and the flat plate portion 5. Therefore, rotary torque of the main body portion 3 is effectively transmitted to the flat plate portion 5. Accordingly, the distal portion of the wire 1 can face in the intended direction. In addition, a pushing force of the main body portion 3 is effectively transmitted to the flat plate portion 5. As a result, excellent blood vessel followability, pushability, and trackability of the wire 1 can be improved.

As shown in FIG. 1, the coil portion 6 is a coil which is disposed so as to cover the distal side of the core portion 2A and is obtained by forming strands in a spiral shape. The coil may be either a so-called densely wound coil in which adjacent strands are in contact with each other or a coil in which adjacent strands are separated from each other. In addition, the distal side of the coil portion 6 can be fixed to the core portion 2A (flat plate portion 5) using the fixation material (fixation portion) 72.

The materials constituting the strands are not particularly limited, but are preferably metal materials such as stainless steel or Pt—Ni alloy. In addition, the size of the coil portion 6 is not particularly limited, and varies depending on use purpose of the wire 1. In the wire 1 used for PTCA, it is preferable that the coil outer diameter of the coil portion 6 is, for example, 0.2 mm to 0.5 mm and the coil length is 10 mm to 1000 mm. The coil outer diameter is preferably constant in the length direction of the wire 1, but may be decreased toward the distal side of the wire 1.

The coil portion 6 may be obtained by combining two or more metal materials. For example, the coil portion 6 may include a first coil portion 61 formed of stainless steel strands on the proximal side; and a second coil portion 62 formed of Pt—Ni alloy strands as radiopaque materials on the distal side, and both coil portions 61 and 62 may be joined through welding or adhering in a boundary portion 63 between the first coil portion 61 and the second coil portion 62. Accordingly, it can be relatively easy for the distal side of the wire 1 to be visually checked under X-ray fluoroscopy.

Next, a modification example of the first embodiment of the wire 1 of the present disclosure will be described.

As shown in FIG. 1, in the wire 1, it is sufficient for the core portion 2A and the coil portion 6 to be fixed to each other in one site on the distal side, but the core portion 2A and the coil portion 6 are preferably fixed to each other in a plurality of sites.

For example, as shown in FIG. 1, in the wire 1, the distal side of the core portion 2A (flat plate portion 5) and the distal side of the coil portion 6 (second coil portion 62) are fixed to each other using the fixation material (fixation portion) 72; a site (the proximal side of the transition portion 4, the small-diameter portion 35, and the distal side of the second tapered portion 34) in the middle of the core portion 2A and a site (boundary portion 63) in the middle of the coil portion 6 are fixed to each other using a fixation material (fixation portion) 73; and a site (the proximal side of the middle-diameter portion 33 and the distal side of the first tapered portion 32) in the middle of the core portion 2A and the proximal side of the coil portion 6 (first coil portion 61) are fixed to each other using a fixation material (fixation portion) 71.

Here, the fixation materials (fixation portions) 71, 72, and 73 can be solder (brazing materials) or adhesives. Note that, in the fixation method of the core portion 2A and the coil portion 6, it is not limited to use the fixation materials 71, 72, and 73, and the fixation portions 71, 72, and 73 may be formed through welding.

As shown in FIG. 1, the wire 1 preferably includes a resin covering portion 8 which is formed so as to cover at least the surface of the coil portion 6 on the distal side (or portion).

Specifically, the resin covering portion 8 preferably covers a part of the surface of the wire or the entirety of the surface of the wire, that is, the entire surface of the second coil portion 62, the entire surface of the coil portion 6 (the first coil portion 61, the boundary portion 63, and the second coil portion 62), or the entire surface of a site on the proximal side of the coil portion 6 and the core portion 2A.

The resin covering portion 8 is preferably made of resin materials such as a fluorine resin, a maleic anhydride polymeric material, and polyurethane. In addition, the thickness of the resin covering portion 8 is preferably, for example, 0.001 mm to 0.05 mm. Since the wire 1 is covered by such a resin covering portion 8, the frictional resistance (sliding resistance) of the wire 1 is decreased, and the operability in a blood vessel is improved.

Next, a second embodiment of a guide wire according to the present disclosure will be described.

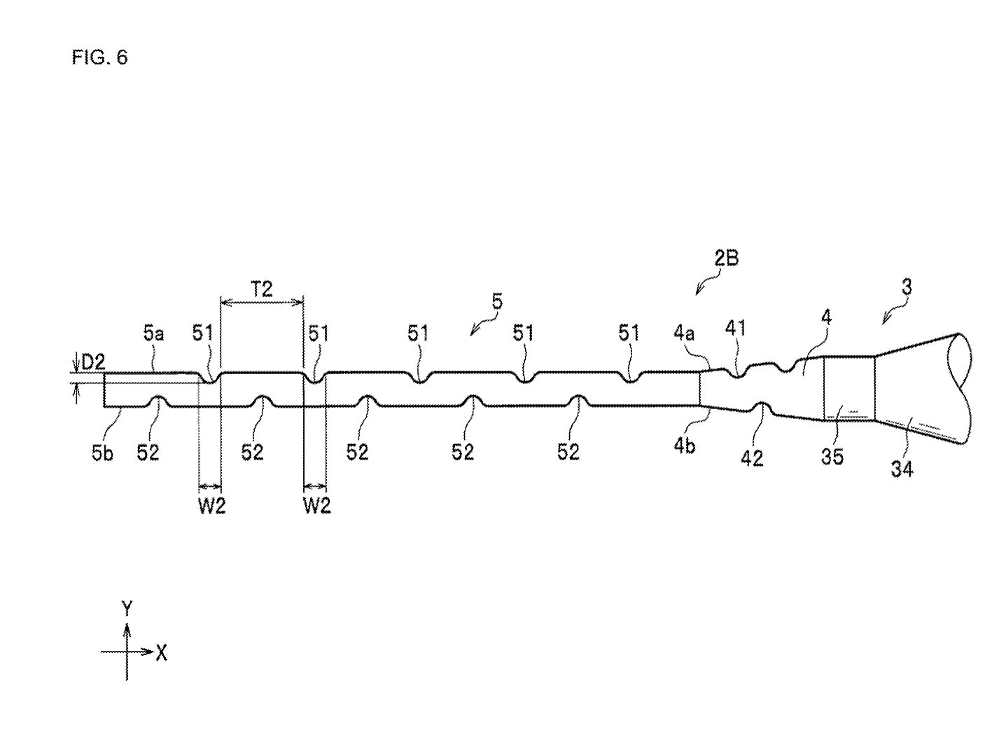

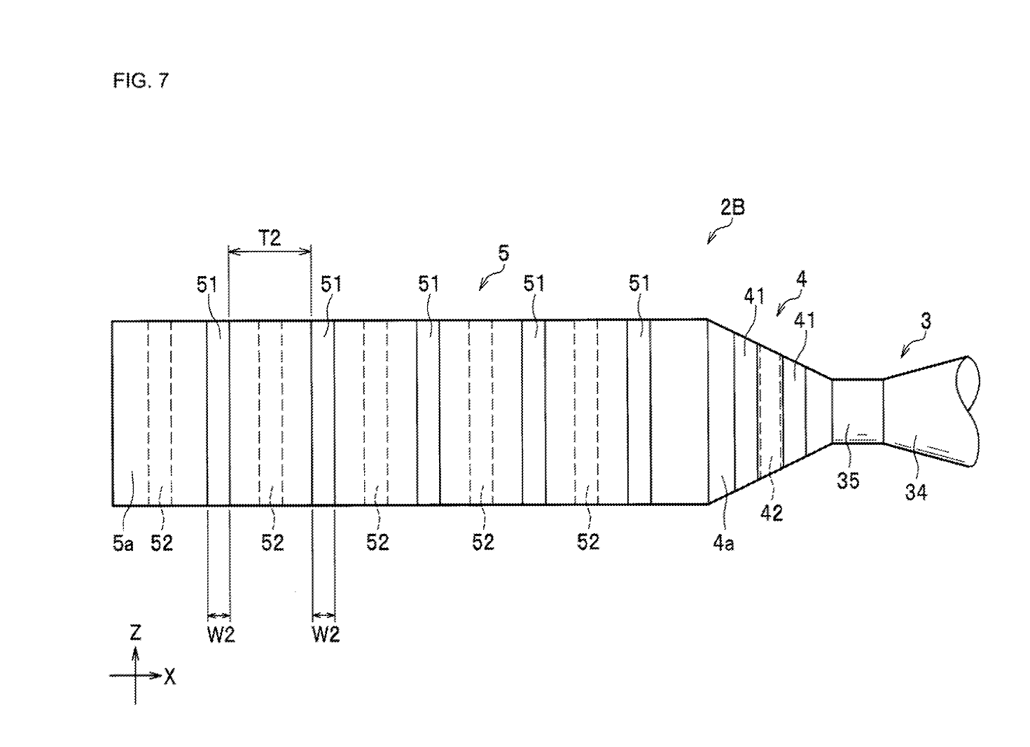

In the guide wire, a core portion 2B (refer to FIGS. 6 and 7) is used instead of the core portion 2A (refer to FIG. 1) of the first embodiment. Therefore, at least one of a groove portion 51 or a groove portion 52 extending in a direction different from a length direction is formed in a flat plate portion 5 in the length direction in addition to forming the groove portion 41 or the groove portion 41 and the groove portion 42 in the transition portion 4. In addition, the groove portion 52 may not be formed. The groove portions 51 and 52 are preferably formed by pressing a mold having a surface of a mold, in which convex portions with a shape similar to the groove portions 51 and 52 are formed, on the surface of the flat plate portion 5. Note that the configuration of the second embodiment other than the groove portions 51 and 52 are the same as described above, and therefore, only the groove portions 51 and 52 will be described and the description of other portions will not be repeated.

As shown in FIGS. 6 and 7, the groove portion 51 is formed on an upper surface 5a of the flat plate portion 5. Note that, although not shown in the drawing, the groove portion 51 may be formed on a lower surface 5b of the flat plate portion 5. Here, the upper surface 5a and the lower surface 5b are surfaces, which become an inner peripheral surface and an outer peripheral surface when a distal portion of the wire is curved. In addition, the direction in which the groove portion 51 is formed is not particularly limited, but is preferably a plate width direction (a direction orthogonal to the length direction) as shown in FIG. 7. Note that, although not shown in the drawing, the groove portion 51 may be formed in an oblique line shape in a direction inclined to the plate width direction at a predetermined angle. Since the oblique line-shaped groove portion 51 is formed in this manner, transmission of rotary torque from a proximal side to a distal side varies depending on the rotational direction of the wire 1 (core portion 2B), and the rotary torque can be relatively easily transmitted in the rotational direction opposite to the inclination direction of the oblique line-shaped groove portion 51. As a result, the blood vessel followability of the wire 1 is further improved. Furthermore, the planar shape of the groove portion 51 is preferably a linear shape as shown in FIG. 7, but may be a polygonal line shape or a curved shape.

It is preferable that the transverse cross sectional shape of the groove portion 51 formed in the flat plate portion 5 is an approximately semi-circular shape. However, the transverse cross sectional shape of the groove portion 51 may be other shapes, for example, an approximately U-shape, an approximately V-shape, or an approximately rectangular shape. The number of groove portions 51 is preferably, for example, 1 to 500. The groove width W2 of the groove portion 51 is preferably, for example, 0.06 mm to 0.5 mm. The groove depth D2 of the groove portion 51 is preferably, for example, 0.001 mm to 0.03 mm. In addition, in a case of forming a plurality of groove portions 51, the groove width W2 is preferably formed to be constant, but may be formed so as to be increased or decreased toward the distal side. The groove depth D2 is also preferably formed to be constant, but may be formed so as to be increased or decreased toward the distal side.

An interval T2 of adjacent groove portions 51 is preferably, for example, 0.1 mm to 2 mm. The plurality of groove portions 51 are preferably formed at even intervals, but may be formed so that the interval T2 increases or decreases toward the distal side. In addition, the plurality of groove portions 51 may have an interval T2 of, for example, 0 mm, that is, may be continuously formed. Furthermore, the groove portions 51, which have been continuously formed, may be formed in a part or all of the upper surface 5a or the lower surface 5b of the flat plate portion 5.

As shown in FIGS. 6 and 7, in the flat plate portion 5, it is preferable that the groove portion 51 is formed on the upper surface 5a and the groove portion 52 is formed on the lower surface 5b of the flat plate portion 5. In addition, it is preferable that the groove portion 51 and the groove portion 52 are mutually alternately disposed in the length direction of the flat plate portion 5. However, the groove portion 51 and the groove portion 52 may be disposed at the same position as each other. In addition, although not shown in the drawing, in a case where the groove portion 51 is formed on the lower surface 5b, the groove portion 52 is formed on the upper surface 5a.

The formation direction, the planar shape, the transverse cross-sectional shape, the number of grooves, the groove width, and the groove depth of the groove portion 52, and the interval between adjacent groove portions 52 are the same as those of the groove portion 51, and therefore, the description thereof will not be repeated. In addition, the formation direction or the like of the groove portion 52 is preferably the same as that of the groove portion 51, but may be different from that of the groove portion 51.

Although not shown in the drawing, in a case where a plurality of groove portions 51 are formed, other groove portions which communicate with two or more arbitrary groove portions 51 in the length direction of the flat plate portion 5 may be formed. In addition, even in a case where a plurality of groove portions 52 are formed, other groove portions which communicate with two or more arbitrary groove portions 52 in the length direction of the flat plate portion 5 may also be formed.

In the guide wire of the present disclosure, the rigidity of the flat plate portion 5 is further decreased by forming the groove portions 51 and 52. Therefore, the flexibility of the distal portion of the guide wire is further improved and the risk of perforating a blood vessel can be reduced. Thus, the safety is improved. Accordingly, the blood vessel followability of the guide wire is further improved.

Modification examples of the second exemplary embodiment of the guide wire of the present disclosure can include an example in which the core portion 2B and the coil portion 6 are fixed to each other in a plurality of sites and an example in which the surface of the wire is covered with the resin covering portion 8, similarly to the first exemplary embodiment.

Next, a third exemplary embodiment of a guide wire of the present disclosure will be described.

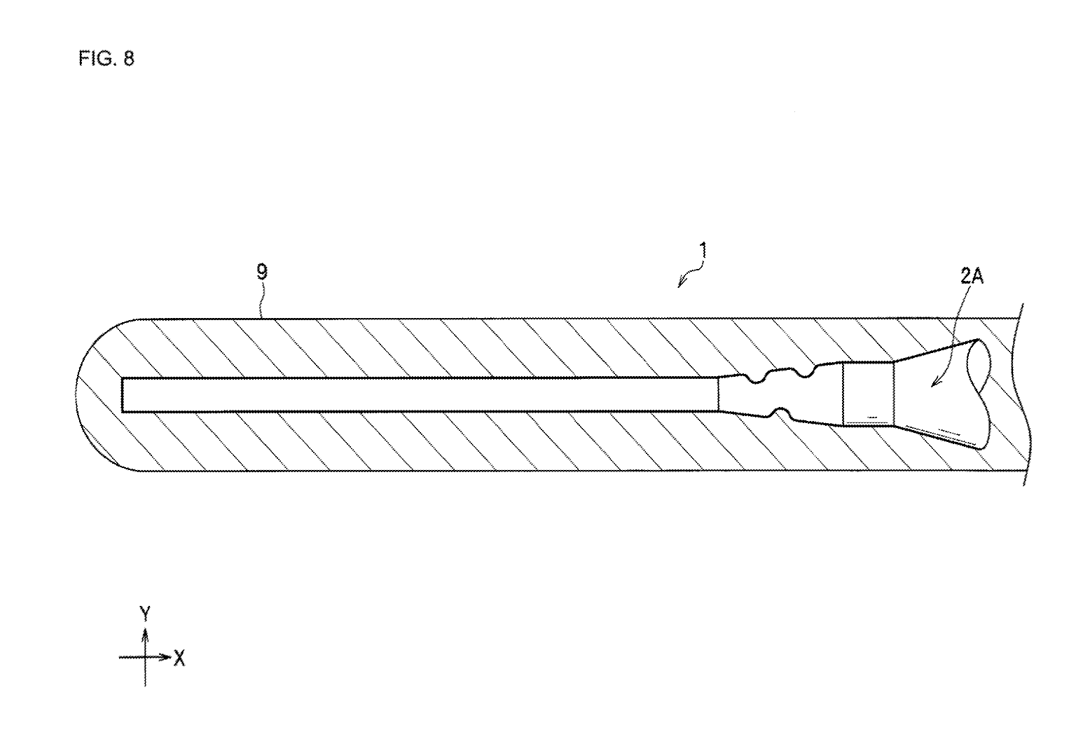

As shown in FIG. 8, a guide wire 1 can include a resin covering portion 9 instead of the coil portion 6 in the first embodiment which includes the core portion 2A. In addition, although not shown in the drawing, the guide wire 1 may include the resin covering portion 9 instead of the coil portion 6 in the second embodiment which includes the core portion 2B (refer to FIG. 6).

Note that the configuration of the third exemplary embodiment other than the resin covering portion 9 is the same as described above. Therefore, only the resin covering portion 9 will be described and the description of other portions will not be repeated.

In accordance with an exemplary embodiment, the resin covering portion 9 is formed so as to cover a distal side of the core portion 2A or the core portion 2B and is made of a resin material. Examples of the resin material include a fluorine resin or polyurethane, and polyurethane is preferable. It is preferable that the resin covering portion 9 is formed such that the thickness of the resin covering portion 9 on the distal side is thicker than that on the proximal side. In accordance with an exemplary embodiment, the thickness of the resin covering portion 9 is preferably, for example, 10 μm to 400 μm. In addition, the resin covering portion 9 is preferably formed such that the distal side of the core portion 2A is rounded.

Since such a resin covering portion 9 is provided, the core portion 2A or the core portion 2B can be prevented from damaging a blood vessel wall when using the guide wire 1. Therefore, the safety is improved. In addition, the frictional resistance (sliding resistance) is decreased in the guide wire 1, and therefore, the operability within a blood vessel is also improved.

Next, a method for using the guide wire of the present disclosure will be described by taking PTCA for an example.

A distal end of the guide wire is inserted into a femoral artery in a state protruding from a distal end of a guiding catheter, through a Seldinger technique, and is inserted into a right coronary artery via an aorta, an aortic arch, and a right coronary artery orifice. Only the guide wire is made to pass a stenosed site of a blood vessel by being further advanced within the right coronary artery while leaving the guiding catheter at a position of the right coronary artery orifice. Then, the distal end of the guide wire stops at a position beyond the stenosed site of the blood vessel. Accordingly, the passage of a balloon catheter for widening the stenosed site is secured.

Next, a distal end of the balloon catheter, which has been inserted from a proximal side of the guide wire, is made to protrude from the distal end of the guiding catheter, and is inserted into the right coronary artery from the right coronary artery orifice by being further advanced along the guide wire. The distal end of the balloon catheter stops at a position at which a balloon of the balloon catheter reaches a position of the stenosed site of the blood vessel.

Next, the stenosed site of the blood vessel is widened by dilating the balloon after injecting a fluid for dilating a balloon into the balloon catheter from the proximal side thereof. By doing this, deposits such as cholesterol which have been adhered to and deposited in the stenosed site of the blood vessel are physically widened, and therefore, interruption of blood flow is resolved.

The fluid for dilating a balloon is removed from the balloon to deflate the balloon. Next, the balloon catheter, the guide wire, and the guiding catheter are removed from the blood vessel by moving the balloon catheter in the proximal direction along with the guide wire. Accordingly, the procedure of PTCA finishes.

The detailed description above describes a guide wire used when guiding a catheter into a lumen in a living body, in particular, a blood vessel. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A guide wire, the guide wire comprising:
a core portion formed of an elongated object having flexibility, wherein the core portion includes a main body portion formed on a proximal side, a flat plate portion formed on a distal side, and a transition portion which connects the main body portion and the flat plate portion, and
at least one groove portion extending in a direction different from a length direction of the transition portion on a slope of the transition portion in the length direction of the transition portion.

2. The guide wire according to claim 1, comprising:
a coil portion which is disposed so as to cover a distal portion of the core portion and is obtained by forming strands in a spiral shape, and the core portion and the coil portion are fixed to each other on the distal portion.

3. The guide wire according to claim 1, comprising:
a resin covering portion which is formed so as to cover a distal portion of the core portion and is made of a resin material.

4. The guide wire according to claim 1, comprising:
at least one groove portion extending in a direction different from a length direction of the flat plate portion on at least an upper surface or a lower surface of the flat plate portion in the length direction of the flat plate portion.

5. The guide wire according to claim 1, wherein the at least one groove portion on the slope of the transition portion comprises at least two groove portions on a same slope or a different slope of the transition portion.

6. The guide wire according to claim 5, wherein the at least two groove portions are formed at equal intervals.

7. The guide wire according to claim 1, wherein the transition portion has four slopes, which connect to surfaces of the flat plate portion.

8. The guide wire according to claim 7, wherein the at least one grooved portion on the transition portion comprises at least one groove in two or more of the four slopes of the transition portion.

9. The guide wire according to claim 1, wherein the different direction of the at least one groove portion on the transition portion is a directional orthogonal to the length direction of the transition portion.

10. The guide wire according to claim 1, wherein the main body portion has a circular transverse cross section shape.

11. The guide wire according to claim 1, wherein the main body portion has a large-diameter portion having a constant outer diameter from a proximal side to a distal side, a first tapered portion of which an outer diameter is decreased toward the distal side, a middle-diameter portion having a constant outer diameter, a second tapered portion of which an outer diameter is decreased toward the distal side; and a small-diameter portion having a constant outer diameter.

12. The guide wire according to claim 4, wherein the at least one groove portion on the at least an upper surface or the lower surface of the flat plate portion in the length direction comprises at least one groove portion on the upper surface and at least one groove portion on the lower surface of the flat plate portion.

13. The guide wire according to claim 4, wherein the at least one groove on the transition portion and the at least one groove portion on the at least the upper surface or the lower surface are on a same surface side.

14. The guide wire according to claim 1, wherein the flat plate portion has a rectangular shape in a transverse cross section.

15. The guide wire according to claim 14, wherein a width of the flat plate portion decreases or increases towards a distal end of the guide wire.

16. The guide wire according to claim 14, wherein a thickness of the flat plate portion decreases or increases towards a distal end of the guide wire.

17. A guide wire formed of an elongated object having flexibility, the guide wire comprising:
a main body portion formed on a proximal side, the main body portion has a large-diameter portion having a constant outer diameter from a proximal side to a distal side, a first tapered portion of which an outer diameter is decreased toward the distal side, a middle-diameter portion having a constant outer diameter, a second tapered portion of which an outer diameter is decreased toward the distal side; and a small-diameter portion having a constant outer diameter;
a flat plate portion formed on a distal side, the flat plate portion having at least one groove portion extending in a direction different from a length direction of the flat plate portion on at least an upper surface or a lower surface of the flat plate portion in the length direction of flat plate portion; and
a transition portion which connects the main body portion and the flat plate portion, the transition portion having at least one groove portion extending in a direction different from a length direction of the transition portion on a slope of the transition portion in the length direction of the transition portion.

18. The guide wire according to claim 17, comprising:
a coil portion covering a distal portion of the guide wire and is obtained by forming strands in a spiral shape, and wherein the guide wire and the coil portion are fixed to each other on the distal portion.

19. The guide wire according to claim 18, comprising:
a resin covering portion covering at least a distal portion of the coil portion.

20. A guide wire, the guide wire comprising:
a core portion formed of an elongated object having flexibility, wherein the core portion includes a main body portion formed on a proximal side, a flat plate portion formed on a distal side, and a transition portion which connects the main body portion and the flat plate portion, and
at least one groove portion extending orthogonal to a length direction of the transition portion on a slope of the transition portion.
